(19) United States
(12) Patent Application Publication
(10) Pub. No.: US 2008/0281187 A1
(43) Pub. Date: Nov. 13, 2008

(54) Ferromagnetic threat detection method apparatus

(75) Inventors: R. Kemp Massengill, Leucadia, CA (US); Richard J. McClure, San Diego, CA (US); Frederick J. Jeffers, Escondido, CA (US)

Correspondence Address: GERALD W. SPINKS, 103 EDWARDS STREET, ABBEVILLE, LA, 70510, US

(73) Assignee: MedNovus, Inc., Leucadia, CA (US)

(21) Appl. No.: 11/975,372

(22) Filed: Oct. 17, 2007

Related U.S. Application Data
(60) Provisional application No. 60/852,574, filed on Oct. 18, 2006.

(51) Int. Cl. A61B 5/05

(52) U.S. Cl. 600/424

(57) ABSTRACT

A method and apparatus for detecting ferromagnetic threat objects on a recumbent patient, including magnetizing/sensing stations having mutually orthogonal magnetizing axes. Two magnetizing/sensing stations can be used, but three or more are preferred. The magnetizing/sensing stations are arranged on a table-like mounting structure providing a path for the patient to roll beneath the magnetizing/sensing stations on a gurney. Three additional magnetizing/sensing stations can be provided on either side, or on each side, of the gurney path, at the same height as the patient.

### CROSS REFERENCE TO RELATED APPLICATIONS

This application relies upon U.S. Provisional Patent Application No. 60/852,574, filed on Oct. 18, 2006, and entitled “Ferromagnetic Threat Detector Method and Apparatus.”

### STATEMENT REGARDING FEDERALLY SPONSORED RESEARCH OR DEVELOPMENT

The U.S. Government has a paid up license in this invention and the right in limited circumstances to require the patent owner to license others on reasonable terms as provided for by the terms of Phase I SBIR Grant No. 1 R43 EB007859-01 awarded by the National Institutes of Health.

### BACKGROUND OF THE INVENTION

1. Field of the Invention

This invention is in the field of screening devices, which are used for preventing ferromagnetic or magnetic objects from being in the vicinity of an operating magnetic resonance imaging apparatus.

2. Background Art

Magnetic resonance imaging (MRI) has been called the most important development in medical diagnosis since the discovery of the x-ray 100 years ago. Magnetic resonance imaging has significant risks, however, and these are becoming more apparent as the number of MRI procedures increases dramatically. In one type of risk scenario, ferromagnetic objects can be propelled rocket-like toward the magnetic resonance imaging magnet by the strong magnetic field of the magnet, sometimes with catastrophic results. This attraction of ferromagnetic objects to the MRI magnet is termed the “missile threat.”

Not only have there been numerous injuries to patients, including one tragic death, but damage to the MRI magnet itself is also a significant problem. One missile-threat accident involved a bobby pin becoming impaled in the nasal passages of a patient, requiring surgical extirpation. A leading expert identifies the missile threat as the number one problem associated with magnetic resonance imaging.

In another type of risk scenario, implanted magnetizable objects brought near an MRI magnet can also cause serious harm to a patient if they move when subjected to the huge magnetic field of the MRI magnet. Aneurysm clips and implanted heart pacemakers are examples of the latter type of threat object.

Magnifying the threat potential for serious harm in either type of risk scenario is the fact that the next generation of MRI magnets are even more powerful than current magnets, generating magnetic fields of 3.0 Tesla, or 30,000 Gauss, or even higher, as opposed to today's “standard” of 1.5 Tesla, or 15,000 Gauss.

In an effort to provide safety, MRI centers have attempted to utilize conventional metal detectors, such as those used for airport security and other security applications. However, conventional metal detectors alarm not only on ferromagnetic threat objects, but also on non-ferromagnetic metallic objects, which are not threats in the magnetic resonance imaging environment. The large number of false positive alarms generated by conventional metal detectors has caused such consternation for MRI staff technologists that conventional metal detectors have been abandoned for this application. Indeed, conventional metal detectors may have no current usefulness as a practical solution for MRI safety.

Because of the inadequacy of conventional metal detectors for MRI safety screening, ferromagnetic-detection portals have been developed, in order to detect ferromagnetic threat objects in the magnetic resonance imaging environment, to minimize the chance of dangerous accidents, and to minimize the number of false positive alarms.

Some patients might walk on their own into the MRI magnet room, some might be brought into the MRI room in a wheelchair, and some might be brought in on a special non-magnetizable “MRI-safe” gurney. Some of the patients who are transported on gurneys are on the gurney because they are unconscious. Obviously, these patients cannot be asked about the presence of any implanted objects, many of which can be rather small and hard to detect. Furthermore, patients who must be transported into the MRI magnet room on a gurney are generally sicker than patients who are ambulatory, with a commensurate limitation of their ability to answer questions about the presence of any implanted objects. Frequently, patients on a gurney have significant memory disturbances and are poor historians, often not remembering major surgical procedures, such as abdominal surgery, orthopedic surgery, and even neurosurgical procedures of the brain, which sometimes include the placement of a ferromagnetic aneurysm clip. The possibility that a patient who must be transported on a gurney has a ferromagnetic pacemaker or other ferromagnetic biostimulation device is also greatly increased, relative to a young and healthy ambulatory patient, thereby greatly increasing the risk of MRI for a patient who must be transported on a gurney.

Therefore, it is especially important that patients being transported on gurneys be subjected to efficient and effective ferromagnetic detection before undergoing magnetic resonance imaging, and the present invention provides an apparatus and a method to accomplish this.

### BRIEF SUMMARY OF THE INVENTION

As used herein, the expression “axis of the magnetic field” refers to the primary axis of the magnetic field, i.e., the orientation of the magnetizing dipole. The present invention's recumbent ferromagnetic threat detector utilizes three detection stations, underneath which a patient on a gurney is successively passed, in order to magnetize and detect a ferromagnetic threat object that might be present on, under, or within a recumbent patient, before the patient undergoes magnetic resonance imaging. Each detection station has a magnetization source, such as a magnet, and a sensing apparatus, such as one or more sensors. Herein, these detection stations are also called magnetizing/sensing stations.

A significant risk of non-detection occurs in the case of an elongated, slender ferromagnetic threat object, when the axis of the threat object is oriented perpendicular to the magnetizing field. When this type of threat object is oriented in this fashion, the magnetizing field does not significantly magnetize the threat object, resulting in little or no chance that the threat object will be detected by a sensor. So, the magnetic field of each magnetizing/sensing station is directed perpendicular to the magnetic fields of the other two magnetizing/sensing stations. Thus, when a patient on a gurney is passed under the recumbent ferromagnetic threat detector of the present invention, any threat object on or within the recumbent patient will be successively exposed to all three mutually orthogonal magnetic fields. This ensures that there will be at least one magnetizing field to which the threat object is not orthogonally oriented. Thus, even for an elongated, slender ferromagnetic threat object, regardless of the orientation of the threat object, the threat object will be magnetized and detected by at least one station. Each magnetizing/sensing station has its own sensors, which are appropriately positioned and configured in a gradiometer format to reduce unwanted false alarms from distant ferromagnetic threats, such as moving elevators or moving chairs. As an alternative, additional magnetizing/sensing stations can be positioned on either side, or on both sides, of the path through which the gurney will pass. As a further alternative to three stations, the present invention can employ only two magnetizing/sensing stations, with the magnetizing source of each station providing a magnetic field directed orthogonal to the other, with the use of magnetization by the off-axis portion of the magnetizing field, to ensure detection of a threat object which may have its major axis oriented orthogonal to the axes of the two magnetic fields.

The novel features of this invention, as well as the invention itself, will be best understood from the attached drawings, taken along with the following description, in which similar reference characters refer to similar parts, and in which:

### BRIEF DESCRIPTION OF THE SEVERAL VIEWS OF THE DRAWINGS

FIG. 1 is a perspective view of the preferred embodiment of the apparatus of the present invention;

FIGS. 2A through 2C are schematic views of three of the possible orientations of threat objects relative to the orientations of the magnetizing/sensing stations used in the embodiment shown in FIG. 1;

FIG. 3 is a schematic view of the magnetic field flux lines generated by a magnet, such as one which might be used in the embodiment shown in FIG. 1;

FIG. 4 is an elevation view of a second embodiment of the apparatus of the present invention, with only two magnetizing/sensing stations;

FIG. 5 is a schematic view of a magnetizing/sensing station as used in the various embodiments of the present invention;

FIG. 6 is an elevation view of a third embodiment of the apparatus of the present invention, with three additional magnetizing/sensing stations arranged to one side of the gurney path; and

FIG. 7 is a plan view of a fourth embodiment of the apparatus of the present invention, with six additional magnetizing/sensing stations arranged on both sides of the gurney path.

### DETAILED DESCRIPTION OF THE INVENTION

Although some ferromagnetic threat objects have inherent magnetization to one degree or another, most ferromagnetic threat objects have virtually no magnetic moment of their own. Hence, they generate almost no magnetic field and are very difficult to detect with a passive detection system. Rather, they must be magnetized with an external source, so that they can be detected. When using an external source of magnetization, such non-magnetic threat objects may still be difficult to detect if the external source is small, such as the earth's magnetic field or the MRI fringing field outside the magnet room. The larger the magnetizing field, up to several hundred Oersted (Oe), the more the threat object will be magnetized, and thus be more readily detected.

Magnetization of a ferromagnetic object can be accomplished by several modalities, such as by using permanent magnets, or by using coils through which current flows, thereby generating a magnetic field. It should be understood that permanent magnets are intended herein to include both flexible and non-flexible permanent magnets and magnet configurations.

The strength of the magnetic field is directly proportional to the size of the magnet. In other words, for a given ferromagnetic sensor system, the larger the magnet, the greater the magnetic field strength, and the more sensitive the system becomes. Thus, using a larger magnet allows detection of smaller and more distant targets.

The level to which a ferromagnetic threat object is magnetized depends, among other considerations, upon the shape of the threat object and the direction of the magnetic field being applied. Elongated objects such, as a nail, are easily magnetized by a magnetic field oriented along the major, longitudinal, axis of the nail, but they are scarcely magnetized at all by magnetic fields which are perpendicular to the longitudinal axis of the nail. If insufficiently magnetized, a sizeable, and therefore dangerous, ferromagnetic threat object can be missed, courting subsequent injury from an implanted object, or risking a missile-threat catastrophe. For instance, the earth's magnetic field is in only one direction for a particular location on the earth. Using the earth's magnetic field to provide the sole magnetization source for a ferromagnetic threat object often results in non-detection of the threat object, and this is more likely to occur whenever the axis of an elongated, slender ferromagnetic threat object is substantially perpendicular to the direction of the earth's magnetic field at that location. An additional problem is that the earth's magnetic field is very small, typically only 0.5 Oe, which is generally inadequate to magnetize most ferromagnetically hard threat objects to a level sufficient to allow detection of the object's induced magnetic field, even if the direction of magnetization from the earth's magnetic field is optimal.

When a magnet is rotated, its magnetic field at any given point rotates by the same angle, but in the opposite direction. In the case of magnets initially having parallel fields, when a first magnet is rotated by 45 degrees clockwise and a second magnet is rotated 45 degrees counterclockwise, the result is that their respective magnetic fields are oriented 90 degrees from one another. Similarly, a third magnet can be oriented with its magnetic field perpendicular to the magnetic fields of the other two magnets. The present invention utilizes three magnetizing/sensing stations, with the magnet in each station having a magnetic field which is oriented perpendicular to the magnetic fields of the other two magnetizing/sensing stations. Thus, when a patient passes under the recumbent ferromagnetic threat detector of the present invention, any threat object present will be exposed to all three mutually orthogonal fields, and so even an elongated, slender threat object will be magnetized and detected by at least one station, regardless of the orientation of the threat object.

An additional advantage of the present invention is that 3-axis magnetization can be achieved without magnetization from the floor below, as would generally be required to achieve 3-axis magnetization in a portal designed for ambulatory patients. This eliminates the need for the ramp typically used to prevent the patient from stepping on the bottom magnetization source, which can trigger a false alarm or otherwise degrade the portal's performance.

The preferred embodiment of the present invention provides three-axis magnetization via the employment of three successively arranged magnetizing/sensing stations, each with its own magnetization source and ferromagnetic sensor system, with each magnetizing/sensing station being oriented to magnetize along an axis orthogonal to the magnetizing axes of the other two. By providing three mutually orthogonal axes of magnetization (x, y, and z), the probability of successfully finding even an elongated, slender ferromagnetic threat object is greatly enhanced.

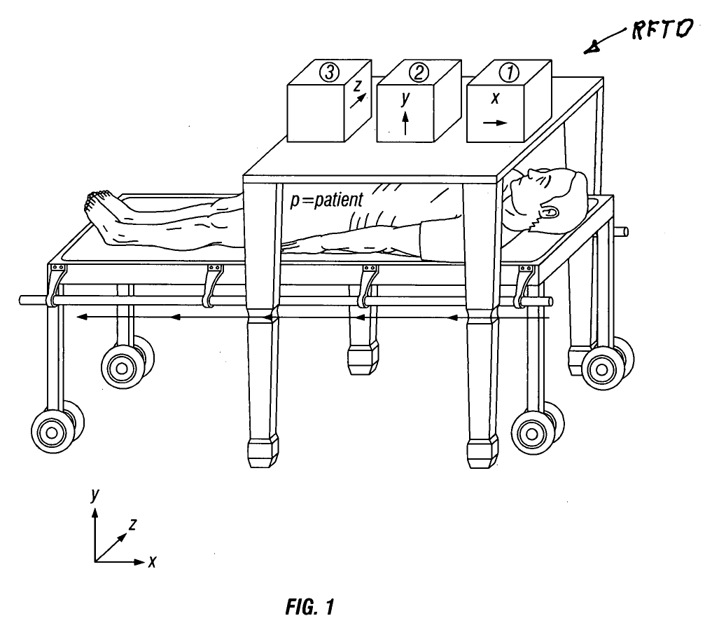

The recumbent ferromagnetic threat detector RFTD of the present invention is ideally suited for a patient who is recumbent, or lying down, on a gurney. In the preferred embodiment, it sequentially places at least three magnetizing/sensing stations in close proximity to the patient as the patient and the gurney pass through the recumbent ferromagnetic threat detector RFTD, beneath the magnetizing/sensing stations. The patient and gurney can pass in either direction along the gurney path. As used in FIG. 1, the x axis is horizontal, and parallel to the path of the gurney; axis y is vertical; and axis z is horizontal, and transverse to the gurney path. However, it should be understood that the axes need not have these relationships to the gurney path or to horizontal and vertical; it is only necessary that the axes be orthogonal to each other. As shown in FIG. 1, a first magnetizing/sensing station 1 affixed on the mounting structure of the recumbent ferromagnetic threat detector RFTD magnetizes any ferromagnetic threat object which may be present, with a field oriented parallel to the x axis. A second magnetizing/sensing station 2 magnetizes any such ferromagnetic threat object, with a field oriented parallel to the y axis. A third magnetizing/sensing station 3 magnetizes any such ferromagnetic threat object, with a field oriented parallel to the z axis. As the gurney transporting the patient is rolled under the recumbent ferromagnetic threat detector RFTD, a first search for ferromagnetic threat objects is carried out by the first magnetizing/sensing station 1, followed by a second search for ferromagnetic threat objects by the second magnetizing/sensing station 2, followed by a third search for ferromagnetic threat objects by the third magnetizing/sensing station 3. Some threat objects will be detected in all three searches. An elongated, slender ferromagnetic object, such as a nail, may be poorly magnetized, and therefore non-detected, in one or more of the magnetizing/sensing stations, if the axis of magnetization in that station is substantially perpendicular to the long axis of the threat object. In that event, however, it will be magnetized and subsequently detected in one or both of the remaining magnetizing/sensing stations. A table-like mounting structure or frame is shown herein, but any other mounting structure capable of positioning the detection stations as described would also suffice.

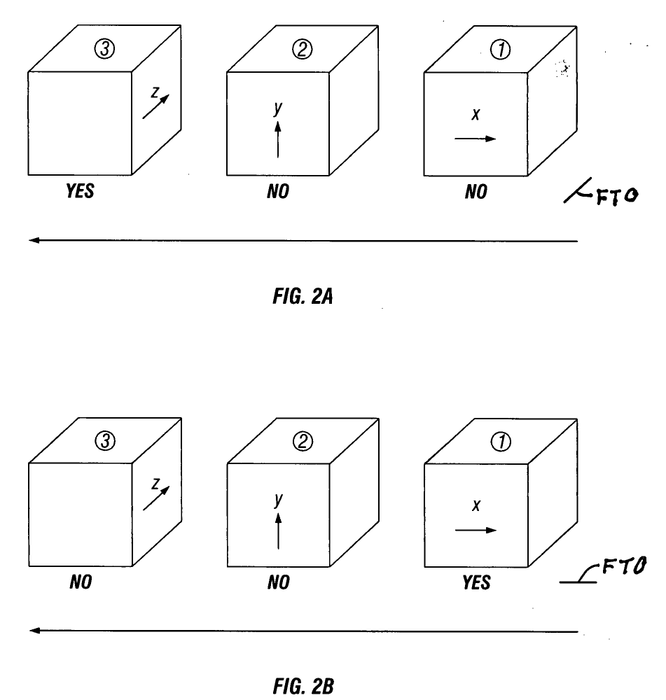

For instance, if an elongated, slender ferromagnetic threat object FTO happens to be aligned parallel to the z axis, as shown in FIG. 2A, the first magnetizing/sensing station 1, which applies a magnetic field substantially parallel to the x axis, which is perpendicular to the axis of the ferromagnetic threat object FTO, will not significantly magnetize the threat object. This will probably result in non-detection of the threat object.

As the gurney continues on its path through the recumbent ferromagnetic threat detector RFTD, the ferromagnetic threat object FTO next encounters the magnetizing field of the second magnetizing/sensing station 2, which is substantially parallel to the y axis, which, again, is at right angles to the axis of the threat object FTO. Very poor magnetization likely will occur, and non-detection will be the likely outcome.

The gurney then continues through the recumbent ferromagnetic threat detector RFTD and encounters the third magnetizing/sensing station 3, which magnetizes substantially parallel to the z direction. As this magnetizing axis is not perpendicular to the axis of the threat object FTO, magnetization and subsequent detection will occur at the third magnetizing/sensing station 3. It should be noted that it is not necessary for the axis of an elongated slender threat object to be parallel to the axis of the magnetizing field for detection to occur. It is only necessary for the axis of such a threat object to not be substantially perpendicular to the axis of the magnetizing field. A magnetizing/sensing station can potentially magnetize and detect any threat object which has a significant dimension which is not perpendicular to the magnetic field generated by that magnetizing/sensing station.

FIG. 2B shows detection of an elongated, slender ferromagnetic threat object FTO aligned parallel to the x axis, which is detected by the first magnetizing/sensing station 1, which magnetizes parallel to the x axis. In this case, the ferromagnetic threat object FTO would not likely be detected by the second and third magnetizing/sensing stations 2, 3. FIG. 2C shows detection of an elongated, slender ferromagnetic threat object FTO aligned parallel to the y axis, which is detected by the second magnetizing/sensing station 2, which magnetizes parallel to the y axis. In this case, the ferromagnetic threat object FTO would not likely be detected by the first and third magnetizing/sensing stations 1, 3.

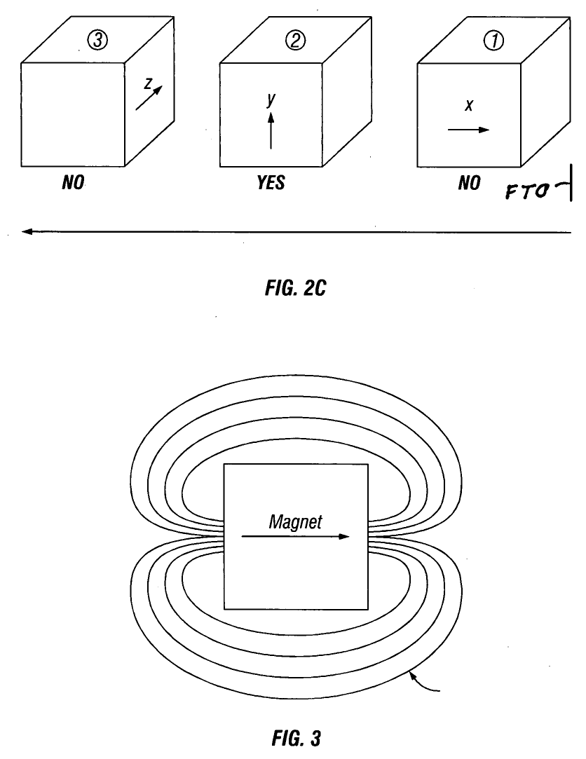

A magnet providing a magnetic field substantially in one axis has divergent field regions according to the magnetic flux lines of the magnet. These divergent field regions are represented by curved flux lines not parallel to the magnet's primary axis of magnetization, and these divergent field regions are smaller in strength than the field along the axis. Magnetization from these divergent (“bloom”) field regions can sometimes result in sufficient magnetization of a ferromagnetic threat object so that detection will occur. FIG. 3 shows the flux lines of magnetic field emanating from a magnetization source. The primary axis of magnetization is indicated by the straight arrow. Note the divergence of this magnetic field into other directions from the primary axis of magnetization. The curved arrow shows a point where the magnetization direction is different from the primary axis of magnetization.

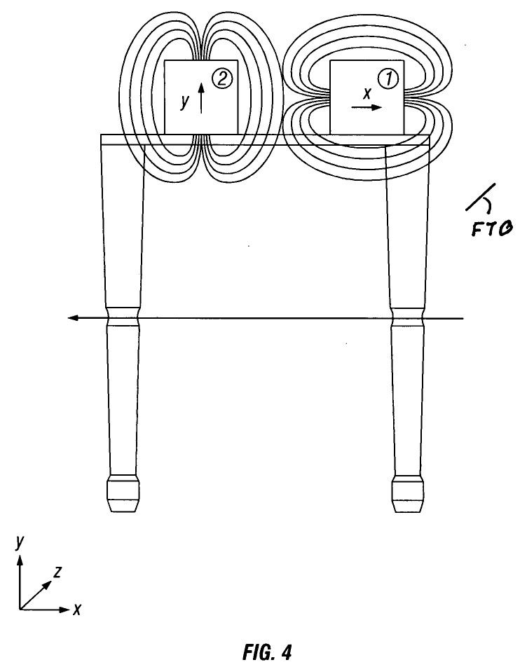

An alternative embodiment of the present invention is to employ only two, rather than three, magnetizing/sensing stations, each magnetizing in a direction orthogonal to the other, as shown in FIG. 4. Magnetization can be provided for the x axis by a first magnetizing/sensing station 1 and for the y axis by a second magnetizing/sensing station 2, but there is no separate provision for a magnetizing/sensing station providing magnetization for the z axis. In this embodiment, an elongated, slender ferromagnetic threat object FTO is detected if at least one of the two magnetic fields is not substantially perpendicular to the longitudinal axis of the threat object, and the threat object projects a significant length along either the x axis or the y axis. If the ferromagnetic threat object is aligned parallel to the z axis, however, off-axis magnetization from the divergent magnetic field regions (the “bloom”) of the magnetization sources oriented parallel to the x axis and the y axis can provide sufficient magnetization of a slender threat object oriented parallel to the z axis to allow detection.

In the preferred embodiment of the present invention, magnetization is achieved via the use of permanent magnets, or permanent-magnet configurations. These can be either flexible, or non-flexible, permanent magnets. Alternatively, configurations and arrays of flexible, or non-flexible, permanent magnets, or combinations thereof, can be employed. In the preferred embodiment, the permanent magnets, or configurations thereof, provide sufficient magnetic field strength to magnetize a small ferromagnetic threat object, such as a bobby pin or an aneurysm clip, at a distance, typically, of 14 to 18 inches from the magnet. Preferably, the magnetic field strength at this distance will be in the range of 20 to 50 Oe. At 8 inches from the magnet, the magnetic field strength will preferably be in the 35 to 70 Oe range.

In a second alternative embodiment, magnetization can be achieved with the use of three successive electromagnetic coils, with one for substantially x-axis magnetization, one for substantially y-axis magnetization, and one for substantially z-axis magnetization. A consideration with coils is that these might be larger and more unwieldy than permanent magnets, when configured to provide the requisite magnetic field strength at the required distance.

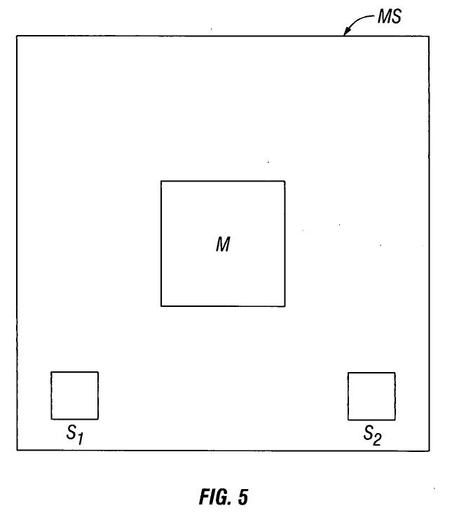

As shown in FIG. 5, the preferred embodiment of the present invention uses a magnet M, and first and second ferromagnetic detection sensors S1, S2 in each magnetizing/sensing station MS. The sensors S1, S2 are arranged in a gradiometer format, as is known in the art, to suppress unwanted signals from distant ferromagnetic objects. The sensors S1, S2 are appropriately and optimally oriented for each magnetizing/sensing station MS of the recumbent ferromagnetic threat detector RFTD, depending upon the sensor type utilized and the direction of magnetization at that particular magnetizing/sensing station MS.

The sensors employed for the present invention detect the threat object's magnetization, i.e., its magnetic field. This magnetization of the threat object is induced by the magnetization source of the present invention, or, in the case of a threat object which is a permanent magnet, the magnetization is pre-existing. The sensors used in the present invention can be of various kinds known in the art. Magneto-resistive sensors, fluxgate sensors, magneto-inductive sensors, magneto-optical sensors, and Hall sensors detect the magnetization of the ferromagnetic threat object whether or not it is in motion. Induction coil sensors also can be used, and these also detect the threat object's magnetization, but only as long as the threat object is in motion, since induction coil sensors do not detect the magnetization of a stationary object. Further, a combination of sensor types can be employed. Saturation-resistant magneto-resistive sensors can also be used. This type of sensor is not affected by off-axis fields, but care must be taken that the on-axis field is not in opposition to that of the sensor's internal bias magnet. The present invention, then, does not limit the type of ferromagnetic detection sensor used.

The patient on a gurney can roll under the recumbent ferromagnetic threat detector RFTD in close proximity to the sensors and the magnetization sources. The strength, at a sensor, of the induced field from a magnetized ferromagnetic threat object is inversely proportional to the cube of the distance between the sensor and the threat object. Similarly, the strength, at the threat object, of the magnetizing field itself is inversely proportional to the cube of the distance between the magnet and the threat object. Therefore, placing the sensors and the magnetization sources in close proximity to any location where a ferromagnetic threat object may be found significantly enhances the detectability of any ferromagnetic threat object that may be present.

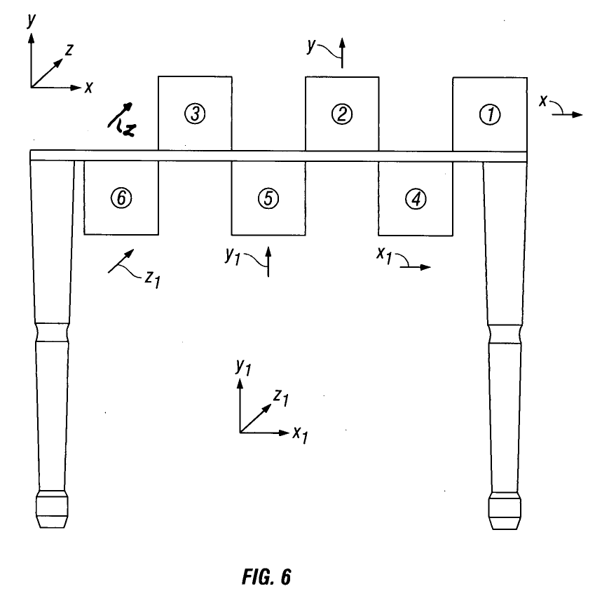

In addition to the first, second, and third magnetizing/sensing stations 1, 2, 3 located on top of the recumbent ferromagnetic threat detector RFTD, which magnetize in three mutually orthogonal axes x, y, z, three additional magnetizing/sensing stations 4, 5, 6 can be positioned on one side of the recumbent ferromagnetic threat detector RFTD. These additional magnetizing/sensing stations 4, 5, 6 can be positioned at the height of the patient from the floor, as shown in FIG. 6. These fourth, fifth, and sixth side magnetizing/sensing stations 4, 5, 6 can be located at positions along the gurney path between the first, second, and third magnetizing/sensing stations 1, 2, 3 which are located above the gurney path, as shown in FIG. 6. The fourth, fifth, and sixth side magnetizing/sensing stations 4, 5, 6 can also magnetize along mutually orthogonal axes x₁, y₁, z₁, respectively. As used in FIG. 6, axes x and x₁ are horizontal, parallel to each other, and parallel to the gurney path; axes y and y₁ are vertical and parallel to each other; and axes z and z₁ are horizontal, parallel to each other, and transverse to the gurney path. The side magnetizing/sensing stations 4, 5, 6 can be placed on either side of the path traveled by the gurney.

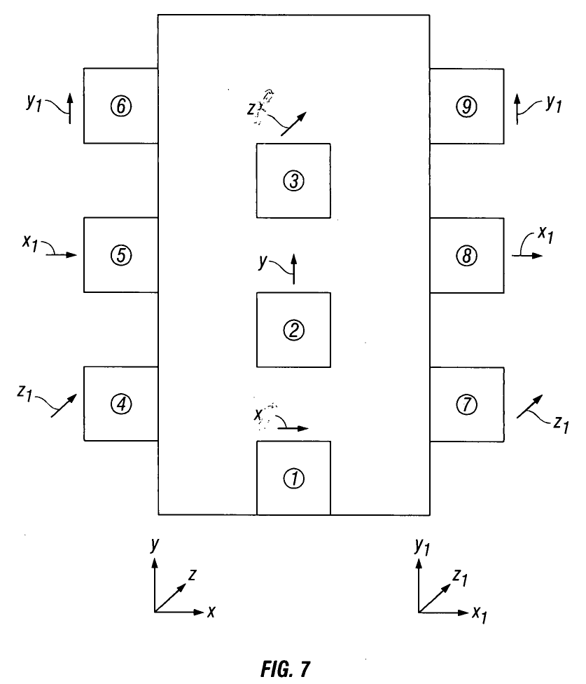

As another alternative, it is possible to place side magnetizing/sensing stations on both sides of the gurney path, as shown in FIG. 7. If the side magnetizing/sensing stations are mounted on both sides of the gurney path, it is important that the direction of magnetization be the same for correspondingly-located magnetizing/sensing station pairs to avoid field cancellation problems in the gurney path. FIG. 7 shows a top view of such an arrangement. It can be seen that the fourth side magnetizing/sensing station 4 corresponds in placement to the seventh magnetizing/sensing station 7, relative to the position along the gurney path. Similarly, the fifth side magnetizing/sensing station 5 corresponds in placement to the eighth magnetizing/sensing station 8, relative to the position along the gurney path. Further, the sixth side magnetizing/sensing station 6 corresponds in placement to the ninth magnetizing/sensing station 9, relative to the position along the gurney path. Each side magnetizing/sensing station in one of these pairs magnetizes in the same direction as its fellow magnetizing/sensing station, to avoid cancellation of the magnetizing field in the center of the gurney path. As used in FIG. 7, axes x and x₁ are horizontal, parallel to each other, and transverse to the gurney path; axes y and y₁ are horizontal, parallel to each other, and parallel to the gurney path; and axes z and z₁ are vertical and parallel to each other. There is one pair of side magnetizing/sensing stations 5, 8 for the x₁ axis, one pair 6, 9 for the y₁ axis, and one pair 4, 7 for the z₁ axis. All 3 of these axes are magnetized by the side magnetizing/sensing stations in this embodiment. Other embodiments can use a single pair of side magnetizing/sensing stations, or two pairs. It is also important that all of the magnetizing/sensing stations be appropriately separated from each other to minimize or eliminate any undesirable field cancellation effects.

While the particular invention as herein shown and disclosed in detail is fully capable of obtaining the objects and providing the advantages hereinbefore stated, it is to be understood that this disclosure is merely illustrative of the presently preferred embodiments of the invention and that no limitations are intended other than as described in the appended claims.

We claim:

1. An apparatus for detecting a ferromagnetic threat object, comprising:
a mounting structure, said mounting structure having a gurney path along which a recumbent patient on a gurney can pass;
a plurality of detection stations on said mounting structure, said detection stations being arranged sequentially along said gurney path;
a magnetic field source in each said detection station; and
at least one magnetic sensor in each said detection station, said at least one sensor being adapted to sense the magnetization of a ferromagnetic threat object in the field established by an associated said magnetic field source in said detection station;
wherein the primary magnetic axis of each said magnetic field source is orthogonal to the primary magnetic axis of at least one other said magnetic field source.

2. The apparatus recited in claim 1, wherein said plurality of detection stations are limited to only two of said detection stations.

3. The apparatus recited in claim 1, further comprising three of said detection stations, each said detection station having the primary magnetic axis of its said magnetic field source oriented orthogonal to the primary magnetic axes of said magnetic field sources at each of the other said detection stations.

4. The apparatus recited in claim 1, wherein said detection stations are arranged above the path of said patient on said gurney passing along said gurney path.

5. The apparatus recited in claim 4, further comprising a second plurality of detection stations on said mounting structure, said second plurality of detection stations being arranged sequentially alongside said gurney path, said second plurality of detection stations being positioned at substantially the same height as said patient on said gurney.

6. The apparatus recited in claim 5, wherein said second plurality of detection stations are mounted along one side of said gurney path.

7. The apparatus recited in claim 5, wherein said second plurality of detection stations are mounted along both sides of said gurney path.

8. A method for detecting a ferromagnetic threat object, comprising:
providing a plurality of detection stations on a mounting structure, each said detection station having a magnetic field source and an associated magnetic sensor, wherein the primary magnetic axis of each said magnetic field source is orthogonal to the primary magnetic axis of at least one other said magnetic field source;
passing a recumbent patient on a gurney along a path, sequentially bringing said patient into close proximity with each of said detection stations;
magnetizing any ferromagnetic threat object present on the patient with a magnetic field established by one of said magnetic field sources; and
sensing, with said magnetic sensor associated with said one magnetic field source, the magnetization of said ferromagnetic threat object in said magnetic field.

9. The method recited in claim 8, further comprising magnetizing said ferromagnetic threat object with an off-axis portion of said magnetic field.

10. The method recited in claim 8, wherein only two of said detection stations are provided.

11. The method recited in claim 8, further comprising:
providing three of said detection stations; and
sequentially bringing said patient into close proximity with each of said three detection stations.

12. The method recited in claim 8, further comprising:
arranging said detection stations above the path of said patient on said gurney; and
sequentially bringing said patient into close proximity beneath each of said detection stations.

13. The method recited in claim 12, further comprising:
providing a second plurality of detection stations on said mounting structure, said second plurality of detection stations being arranged sequentially alongside said gurney path, at substantially the same height as said patient on said gurney; and
sequentially bringing said patient into close proximity beneath each of said first plurality of detection stations and alongside each of said second plurality of detection stations.

14. The method recited in claim 13, wherein said second plurality of detection stations are arranged sequentially along both sides of said gurney path, said method further comprising sequentially bringing said patient into close proximity beneath each of said first plurality of detection stations and between said second plurality of detection stations.
